(19) United States
(12) Patent Application Publication
(10) Pub. No.: US 2013/0171139 A1
(43) Pub. Date: Jul. 4, 2013

(54) NCAM-VASE AND NEURODEGENERATION

(71) Applicants: Jane Louise Saffell, London (GB); Michael Jonathan Delves, London (GB); Alexandra Antoinette Anderson, London (GB)

(72) Inventors: Jane Louise Saffell, London (GB); Michael Jonathan Delves, London (GB); Alexandra Antoinette Anderson, London (GB)

(21) Appl. No.: 13/695,289

(22) PCT Filed: Apr. 28, 2011
(86) PCT No.: PCT/GB11/00659

§ 371 Date: Mar. 12, 2013

(30) Foreign Application Priority Data
Apr. 30, 2010 (GB) 1007428.4

(51) Int. Cl. C12N 15/113; A61K 31/352; C07K 14/705; A61K 45/06; A61K 31/713; A61K 38/17; A61K 39/395; A61K 38/18; C07K 16/28

(52) U.S. Cl. CPC C12N 15/1138; A61K 38/1825; A61K 31/352; C07K 14/70503; C07K 16/2803; A61K 31/713; A61K 38/1774; A61K 39/3955; A61K 45/06
USPC 424/134.1; 435/375; 514/44 A; 424/139.1; 514/8.4; 514/8.3; 536/24.5; 530/387.9; 530/387.3

(57) ABSTRACT

Inhibitors of NCAM-VASE, compositions comprising said inhibitors, and methods of using said inhibitors for stimulation of neuroplasticity and/or neuroregeneration in the central nervous system, and for increasing neuronal cell response to agents that stimulate neuroplasticity and/or neuroregeneration in the central nervous system, are provided. The inhibitor or composition may be used, for example, for treating brain or spinal cord injury; schizophrenia, motor neurone disease; a neurodegenerative disorder such as Alzheimer's disease, multiple sclerosis or Parkinson's disease; ischaemia caused by stroke; or for improving learning and/or memory.

### FIELD OF INVENTION

This invention relates to neurodegenerative disease and to nerve damage in the central nervous system and treatments thereof. More particularly it relates to the promotion of axonal regeneration and survival.

### BACKGROUND TO INVENTION

The need to stimulate neurite outgrowth, which is also called axon regeneration, arises in the treatment of many diseases and injuries of the central nervous system (CNS) including paralysis caused by spinal cord injury, motor neurone disease, neurodegenerative diseases, for example, multiple sclerosis, Alzheimer's disease, and Parkinson's disease, and ischaemia, caused, for example, by stroke.

The stimulation of neurite outgrowth is also thought to be important in increasing neuroplasticity which is thought to be important in memory and learning.

Many agents can stimulate neurite outgrowth in vitro. Growth factors, for example, nerve growth factor (NGF), fibroblast growth factor (FGF), glial cell derived growth factor (GDNF), brain derived growth factor (BDNF), ciliary neurotrophic factor (CNTF) and peptides derived from NCAM as disclosed in Saffell (WO 01/96364) have trophic (cell survival-promoting) and axon regeneration effects, and there is expectation that they may be useful in the treatment of damaged or diseased nervous system. Many of the factors have been, are, or will be in clinical trial.

So far, however, the clinical improvements obtained with some of the above-mentioned materials have been at least partly disappointing. Furthermore, many of the growth factors mentioned above are mitogenic, and entail the risk that they can stimulate tumour formation. There is a need to provide novel agents for stimulating axonal regeneration and neuronal plasticity and regeneration which are either better than the existing agents or which can increase the effectiveness of existing agents allowing them to be used either more effectively or at lower dosages so as to mitigate their side-effects.

Following damage to axons in the central nervous system, for example as a result of CNS diseases or spinal cord injury, neurite outgrowth is inhibited by the milieu of the CNS. In particular, CNS myelin (oligodendrocytes) and glial scars (reactive astrocytes) are known to prevent axon regeneration. Thus, approaches to stimulate CNS regeneration include targeting inhibitory proteins in these tissues.

### NCAM & NCAM-VASE

One of the receptor systems known to control axonal growth involves a homophilically binding cell adhesion molecule called neural-cell adhesion molecule (NCAM). Alternative splicing generates 20 to 30 isoforms of NCAM and there is also evidence that its activity can be modulated by differential glycosylation.

One of the isoforms of NCAM is known as NCAM-VASE and differs from other isoforms of NCAM in that it uses an alternative exon (the “VASE exon”) encoding an additional 10 amino acids in the Ig4 domain.

NCAM molecules consist of 5 Ig (immunoglobin-like) domains and 2 Fn (fibronectin-like) domains together with a transmembrane anchor. NCAM mediates cell adhesion by forming homophilic association between the Ig domains of NCAM expressed on the surface of adjacent cells.

Neuronal contact with NCAM-expressing cells is known to stimulate axon regeneration. However, the VASE sequence inhibits this property of NCAM, since contact of neurons with NCAM-VASE-expressing cells does not stimulate axon regeneration.

Following the observation that the VASE-sequence inhibits NCAM stimulation of axon outgrowth, it was hypothesised that soluble peptides containing the 10 amino acid sequence specific to NCAM-VASE might be useful in disinhibiting the inhibiting activity of endogenous NCAM-VASE.

However, it has been demonstrated that a VASE peptide (a short peptide containing the 10-mer encoded by the VASE-exon) did not reverse the inhibitory effects associated with expression of NCAM-VASE (Saffell et al., J. Cell. Biol., 1994, Vol 125, No. 2, p 427-536). A family of receptor tyrosine kinases known to be activated by FGF show a region that has similarities with the NCAM-VASE 10 amino acid sequence. The FGF receptor is widely expressed in post-mitotic neurons, and neurons respond to activation of the receptor by extending longer neurites. It has been suggested that the 10-amino acid sequence specific to NCAM-VASE might inhibit NCAM-stimulated neurite outgrowth by preventing NCAM from binding to and activating FGF receptors in neurons.

That study taught away from the use of NCAM-VASE inhibitors, because a molecule targeting NCAM-VASE but not targeting other isoforms of NCAM was thought to necessarily target the additional sequence of NCAM-VASE encoded by the VASE-exon, and agents targeting that sequence encoded by the VASE-exon or its interactions were assumed to also disrupt NCAM-stimulated axon outgrowth. This means that NCAM-VASE was thought to be difficult to inhibit without also inhibiting other isoforms of NCAM.

The present invention is based on the discovery that NCAM-VASE can be specifically inhibited by agents which do not disrupt NCAM-stimulated axon outgrowth, and the discovery that contact of neurons with NCAM-VASE-expressing cells prevents them from responding to other axon stimulating agents. Thus, the antagonistic effect that NCAM-VASE has on promoters of neuroplasticity and axon regeneration (including, but not limited to NCAM) can be lifted by use of NCAM-VASE inhibitors with the proposed clinical benefits described herein.

### SUMMARY OF INVENTION

The invention provides an inhibitor of NCAM-VASE for use as a medicament for stimulation of neuroplasticity and/or neuroregeneration in the CNS.

The invention also provides an inhibitor of NCAM-VASE for use as a medicament for increasing neuronal cell response to agents that stimulate neuroplasticity and/or neuroregeneration in the CNS.

The invention also provides a pharmaceutical composition comprising an inhibitor of NCAM-VASE and optionally further comprising a second agent which promotes neuroplasticity and/or neuroregeneration.

The invention also provides a method of stimulating in vitro or in vivo neuroplasticity and/or neuroregeneration comprising providing an inhibitor of NCAM-VASE to neuronal cells and/or tissues.

The invention also provides a method of enhancing neuroplasticity and/or neuroregeneration in a human subject in need thereof comprising providing a therapeutically effective amount of an inhibitor of NCAM-VASE to said subject.

The invention also provides a compound which inhibits NCAM-VASE but which does not inhibit other isoforms of NCAM, said inhibitor selected from the group consisting of, an anti-sense nucleic acid or an interfering nucleic acid capable of selectively reducing the translation of an mRNA having the nucleotide sequence: gcttcgtggactcgaccagagaagcaagag; an antibody or fragment or derivative thereof able to bind to NCAM-VASE; and a soluble protein comprising the Ig4 domain of NCAM-VASE.

The invention also provides a method of identifying a compound for use as a medicament for stimulation of neuroplasticity and/or neuroregeneration in the central nervous system, comprising identifying a compound that is an inhibitor of NCAM-VASE.

The invention also provides a method of identifying an inhibitor of NCAM-VASE comprising culturing neurons on a NCAM-VASE-expressing fibroblast monolayer in the presence of a test compound, culturing neurons on a control NCAM-VASE-expressing fibroblast monolayer in the absence of the test compound, measuring neurite length following culture of the neurons, and comparing neurite length between neurons cultured in the presence of the test compound and neurons cultured in the absence of the test compound, wherein greater neurite length in the neurons cultured in the presence of the test compound indicates the presence of an inhibitor of NCAM-VASE.

The invention also provides a method of screening a subject in need of stimulation of neuroplasticity and/or neuroregeneration in the CNS, for the likelihood of responding to treatment with an inhibitor of NCAM-VASE, comprising analysing a sample that has been obtained from the subject for the presence of NCAM-VASE, wherein greater levels of NCAM-VASE indicates a greater likelihood that the subject will respond to treatment with an inhibitor of NCAM-VASE.

### DESCRIPTION OF THE DRAWINGS

FIG. 1 shows the localisation of a) total NCAM and b) NCAM-VASE in coronal sections of the adult rat brain.

FIG. 2 shows the localisation of a) total NCAM and b) NCAM-VASE in parasigittal sections of adult rat cerebellum.

FIG. 3 shows the localisation of NCAM-VASE in developing cerebellar oligodendrocytes.

FIG. 4 shows the upregulation of NCAM-VASE in reactive astrocytes.

FIG. 5 shows NCAM-VASE is expressed in spinal dorsal root entry zone (DREZ) astrocytes.

FIG. 6 shows NCAM-VASE is expressed in the ependymal cells lining the central canal of the spinal cord and the ventricles of the brain.

FIG. 7 shows exclusion of NCAM-VASE from ependymal cells lining the lateral ventricle and from NCAM-positive neurons in the sub-ventricular zone.

FIG. 8 shows a) NCAM-VASE RNAi sequences, and b) a pan-NCAM RNAi sequence.

FIG. 9 shows that the pan-NCAM RNAi sequence down-regulates NCAM and NCAM-VASE expression.

FIG. 10 shows that a) first and b) second NCAM-VASE RNAi sequences reduce NCAM-VASE expression.

FIGS. 11 and 12 show that NCAM-VASE RNAi sequences down-regulate NCAM-VASE expression without affecting non-VASE NCAM expression.

FIG. 13 shows that culture on NCAM-VASE-monolayers prevents axon regeneration stimulated by soluble NCAM.

FIG. 14 shows that culture on NCAM-VASE-expressing monolayers prevents neurons exposed to axon regeneration agents from extending longer axons.

FIG. 15 shows that soluble NCAM-VASE is not inhibitory to axon outgrowth stimulated by NCAM and can relieve NCAM-VASE monolayer-mediated suppression of axon outgrowth.

FIG. 16 shows that cell-cell adhesion is stronger between NCAM-VASE-expressing cells than between NCAM-expressing cells.

### DEFINITIONS

### Ig4 Domain

An Ig domain is a protein domain consisting of a two-layer sandwich of between 7 and 9 anti-parallel β-strands arranged into two β-sheets with a Greek key topology. The “Ig4 domain” is used herein to designate a sequence having at least 90% identity to the 4^{th} μg domain obtained from any isoform of NCAM from any species. Preferably the species is human.

### NCAM Isoforms

This term includes all isoforms of NCAM including the three most common forms NCAM-120, NCAM-140 and NCAM-180 and all isoforms containing the VASE-exon.

### NCAM-VASE

NCAM-VASE is used herein to encompass any isoform of NCAM which contains a VASE-exon.

### Neuroregeneration

Neuroregeneration is the ability of nerve cells and tissues to restore function lost by injury or degeneration as assessed functionally or by a surrogate assay such as the axon regeneration assays disclosed herein.

### VASE-Exon (Giving Sequence for Rat and Human)

The VASE exon is the exon specific to the VASE isoform of NCAM. The protein and nucleic acid sequences for human and rat VASE-exon are given below:

```
Human
gct tcg tgg act cga cca gag aag caa gag
  A S W T R P E K Q E
Rat
gca tcg tgg act cga cca gag aag caa gag
  A S W T R P E K Q E
```

According to certain embodiments the VASE-exon is the human sequence given above and derivatives therefore which represent silent changes to the nucleic acid sequence.

### DETAILED DESCRIPTION OF THE INVENTION

The invention provides an inhibitor of NCAM-VASE for use as a medicament for stimulation of neuroplasticity and/or neuroregeneration in the CNS. The invention also provides a related method of stimulating neuroplasticity and/or neuroregeneration by providing an inhibitor of NCAM-VASE to neuronal cells and/or tissues.

The invention also provides a compound which inhibits NCAM-VASE but which does not inhibit isoforms of NCAM that do not contain the VASE-exon. For example the inhibitor may be selected from the group consisting of an anti-sense nucleic acid or an interfering nucleic acid capable of selectively reducing the translation of an mRNA having the nucleotide sequence: gcttcgtggactcgaccagagaagcaagag; an antibody or fragment thereof able to bind to NCAM-VASE; and a soluble protein comprising the Ig4 domain of NCAM-VASE.

According to a further aspect, the inhibitor increases neuronal cell response to agents that stimulate neuroplasticity and/or neuroregeneration. Those agents include FGF, NGF, CNTF, GDNF, NT3, NT4/5, BDNF, and agents that mimic or elevate cAMP in neurons (e.g. dbcAMP, forskolin).

The stimulation of neuroplasticity and/or neuroregeneration may be manifest as the stimulation of axon-regeneration or neurite-outgrowth (for example, as assessed in a neurite-outgrowth assay such as that disclosed herein) or it may be manifest as a clinical improvement in a subject or patient).

### Inhibitors

According to certain embodiments, the inhibitor comprises the 10 amino acid sequence encoded by the VASE-exon contained within a larger protein domain, preferably at least a protein domain having at least 80%, preferably at least 90% identity to the Ig4 domain of NCAM-VASE. The Ig4 domain may be part of a larger molecule, for example, it may be part of a molecule containing other Ig or Fn domains of NCAM, for example, the molecule may comprise the Ig3 and Ig4, the Ig4 and Ig5, the Ig3, Ig4 and Ig5 domains of NCAM-VASE. According to certain embodiments the molecule may comprise substantially the whole NCAM-VASE molecule which may optionally be further fused to a “tag” domain such as the Ig Fc domain or a His tag to aid production or purification.

According to certain embodiments, the inhibitor is preferably a soluble polypeptide (i.e. the inhibitor is soluble in water).

According to certain embodiments, the inhibitor is preferably not a peptide having a sequence of less than 25 amino acids, which sequence includes the sequence of 10 amino acids encoded by the VASE-exon More preferably, the inhibitor is not a peptide having a sequence of less than 20 amino acids, still more preferably less than 15 amino acids, which sequence includes the VASE-encoded 10-amino acid sequence. The inhibitor preferably does not interact in a cellular assay with the NCAM receptor to an extent sufficient to result in material disruption of NCAM signalling through the FGF receptor.

A molecule in accordance with the invention (for example a protein molecule) may be derivatised by any standard method known in the art, for example, it may be lipidated, glycosylated or PEGylated.

According to certain other embodiments, the inhibitor may be a small molecule inhibitor. However, in all cases it is important that the inhibitor is specific for NCAM-VASE and does not inhibit other isoforms of NCAM to a material extent (for example, the selectivity must be at least 10:1, preferably at least 100:1, more preferably at least 1000:1).

According to certain embodiments the inhibitor acts at the level of the NCAM-VASE mRNA and is an antisense mRNA targeting the VASE-exon or an interfering RNA (RNAi) targeting the VASE-exon, for example, an interfering RNA having one of the specific sequences (or at least 90% of the sequences) disclosed in the examples herein.

According to certain embodiments the inhibitor is an antibody or antibody fragment or derivative able to bind selectively to NCAM-VASE (preferably with a corrected cross-reactivity for other isoforms of NCAM of less than 1%, more preferably less than 0.1%, still more preferably less than 0.01%). Preferably the antibody or antibody fragment is not targeted to an epitope consisting of the sequence of 10 amino acids encoded by the VASE-exon, or to a short peptide containing that sequence (e.g. a peptide containing up to 15 amino acids). Rather, the antibody or antibody fragment is targeted to other epitopes which exist only in NCAM-VASE and not in other isoforms of NCAM. A suitable antigen comprises the Ig4 domain of NCAM-VASE. A further example of a suitable antigen would be whole NCAM-VASE. Antibodies may be affinity purified against NCAM-VASE and/or negatively selected again non-VASE isoforms of NCAM.

Suitable antibodies and antibody fragments include, for example, intact antibodies (polyclonal, monoclonal, or chimeric), antibody fragments, antibody heavy chains, antibody light chains, single chain antibodies, single-domain antibodies (a VHH for example), Fab antibody fragments, Fc antibody fragments, Fv antibody fragments, F(ab′)₂ antibody fragments, Fab′ antibody fragments, and single-chain Fv (scFv) antibody fragments.

### Combination Therapies

In certain circumstances lifting the inhibition provided by NCAM-VASE may be sufficient to bring about an increase in neuroplasticity or neuroregeneration, perhaps because of the presence of growth factors and other stimulators of axon regeneration in the cellular milieu. In other circumstances it may be preferred to use an inhibitor of NCAM-VASE in combination with a second agent that promotes neuroplasticity and/or neuroregeneration.

This second agent may include any neural growth factor. Specific examples of second agent include NGF, BDNF, FGF, CNTF, GDNF, NT3, NT4/5, and agents that mimic or elevate cAMP (e.g. dbcAMP, forskolin).

The use of NCAM-VASE inhibitors is applicable to use in combination with both agents which act through the FGF receptor (for example, NCAM and peptides thereof) and agents which do not act through the FGF receptor.

The invention encompasses and relates to use of an NCAM-VASE inhibitor in combination with a second agent. The two agents may be administered simulataneously, sequentially or separately (such that they are still able to operably interact). According to certain embodiments the two agents are formulated into the same composition or dosage form. Accordingly the invention provides a pharmaceutical composition comprising an inhibitor of NCAM-VASE according to the invention, and further comprising a second agent as described above.

The invention also encompasses kits containing an inhibitor of NCAM-VASE and a second agent, for simultaneous, sequential or separate administration.

### Methods of the Invention

The methods of the invention may be in vitro methods or in jurisdictions where this is permitted, they may be methods of treatment practised on the human or animal body. In jurisdictions where such methods are not permitted the invention provides inhibitors for use in corresponding methods and use of such inhibitors for the manufacture of a medicament for use in corresponding methods.

### Subjects to be Treated

Aspects of the invention may relate to treatment of humans or non-human mammals. Specifically, treatment may be of non-human mammals that act as models for human disease, for example rodents, in particular, rats, rabbits and mice.

Humans to be treated are preferably those in need of therapy, for example, those who have suffered an injury or disease which may be treated by promoting neurite outgrowth. In certain embodiments, groups or individuals to be treated may be selected for their predisposition to develop a disease (for example, as identified by genetic screening or familial history). In other embodiments the subjects may be selected for treatment based on a prior screen which identifies NCAM-VASE expression which is at a level for which inhibition is indicated. In other cases, subjects selected for treatment may be those who have failed to show satisfactory improvement when given agents such as growth factors which might have been expected to cause a clinical improvement.

### Compositions

In certain embodiments, the pharmaceutical composition of the invention is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilising agent and a local anaesthetic such as lignocaine to ease pain at the site of the injection.

For treatment of neurodegenerative diseases that involve brain degeneration, it is preferable to use a formulation suitable for delivery of the inhibitor across the blood-brain barrier. Examples of suitable formulations are given in “Delivery of peptides and proteins through the blood-brain barrier” Bickel U et al. Advanced Drug Delivery Reviews 46, 247-279.

The amount of the inhibitor of the invention which will be effective in the treatment of a particular disorder or condition will depend on the nature of the disorder or condition, and can be determined by standard clinical techniques. In addition, in vitro assays may optionally be employed to help identify optimal dosage ranges. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal test systems.

### Diseases, Disorders and Injuries

During development of the animal, the ability of neurons to elaborate axons and dendrites in a highly organised manner which is essential for the establishment of appropriate synoptic connections is lost as the animal reaches adulthood.

The present invention is based on the hypothesis that one of the reasons this takes place is because adult cells become refractory to growth signals due to increasing expression of NCAM-VASE, and the fact that NCAM-VASE is high in tissues refractory to axon regeneration, such as CNS myelin, reactive astrocytes and DREZ (dorsal route entry zone) astrocytes.

The various aspects of the invention are therefore suitable for use relating to the treatment of diseases, disorders and injuries for which a return to a developmentally earlier phenotype of greater neuronal potential for plasticity and redevelopment would be beneficial.

Injuries suitable for such treatment include those resulting in paralysis, for example, from trauma and or stroke and also neurodegenerative diseases such as Alzheimer's and Parkinson's disease, multiple sclerosis and motor neurone disease.

Schizophrenia has been associated with aberrant NCAM-VASE expression and so the present invention relates to the treatment of that disorder as well as to treatments to improve memory and learning.

In addition, it is also thought that neutralisation of NCAM-VASE may be important for treating heart disease or ischemia. NCAM-VASE expressed in cardiac (but not skeletal) muscle, and total NCAM (including NCAM-VASE) is known to be upregulated in ischemic cardiomyopathy specifically (see, for example, Gattenlohner et al, 2003, Am. J. Pathol. 163, 1081-1090) The present invention therefore also relates to the treatment of heart disease or ischemia.

### Routes of Administration

Inhibitors and/or compositions of the invention should be administered by a route appropriate for the condition to be treated and for the treatment desired. Such routes include intradermal, intramuscular, intraperitoneal, intravenous, subcutaneous, intranasal, epidural, and oral routes. The peptides may be administered by any convenient route, for example by infusion or bolus injection, by absorption through epithelial or mucocutaneous linings for example, oral mucosa, rectal and intestinal mucosa, and may be administered together with other biologically active agents. Administration can be systemic or local.

According to certain embodiments the inhibitor or composition may be injected directly into the brain capsule, the ventricles or the cerebrospinal fluid. According to certain embodiments a slow release composition or implantable pump may be surgically implanted so as to introduce the inhibitor or composition to the desired site.

If the inhibitor or composition is introduced such that it needs to cross the blood-brain barrier before reaching the target CNS cells or tissues, it may be selected or formulated so as to facilitate this.

### Production of Protein Inhibitors

A protein of the invention may be produced recombinantly or synthesised chemically. A nucleic acid encoding the peptide may be synthesised chemically or obtained from tissue.

For recombinant expression of a protein the nucleic acid encoding the protein can be inserted into an appropriate expression vector, i.e., a vector that contains the necessary elements for the transcription and translation of the inserted coding sequence. In a preferred embodiment, the regulatory elements for example, promotor, are heterologous i.e., not the native gene promotor. Promotors which may be used include the SV40 early promoter (Bernoist and Chambon, 1981, Nature 290: 304-310), and the promoter contained in the 3′ long terminal repeat of Rous sarcoma virus (Yamamoto et al., 1980, Cell 22: 787-797), or the CMV promoter, particularly for use in mammalian cell lines, among others.

A variety of host vector systems may be utilized to express the protein encoding sequence. These include mammalian cell expression systems, including cells infected with virus for example, vaccinia virus and adenovirus; mammalian cell systems with plasmids; insect cell systems infected with virus, for example, baculovirus; microorganisms for example yeast containing yeast vectors; or bacterial transformed with bacteriophage, DNA, plasmid DNA, or cosmid DNA.

When a protein of the invention has been recombinantly expressed, it may be isolated and purified by standard methods including chromatography for example, ion exchange, affinity, and sizing column chromatography, centrifugation, differential solubility, or by any other standard technique for the purification of proteins.

The following non-limiting examples illustrate the invention.

### EXAMPLES

### Example 1

### Distribution of NCAM-VASE in the Central Nervous System

An affinity purified anti-VASE antibody was developed that is specific only for the VASE isoform of NCAM, and which can recognise NCAM-VASE expressed on the surface of cultured cells.

A peptide immunogen consisting of the VASE sequence (underlined), four flanking amino acids, and an N-terminal Cys (CSEEKASWTRPEKQETLDG), coupled to Blue Carrier Protein, was used to produce polyclonal rabbit antiserum. The peptide sequence was that previously used by (Hemperly et al., Exp Neurol, 1998, 154, 1-11) for the same purpose. The crude antiserum was affinity purified using a column containing NCAM-VASE covalently coupled to sepharose beads to isolate anti-VASE antibodies that recognised the sequence in its native form in the whole NCAM-VASE ectodomain. (The NCAM-VASE (with a C-terminal-Fc tag) was produced in mammalian CO7 cells transfected with a plasmid vector encoding NCAM-VASE-Fc. It was affinity purified from the culture medium into which it was secreted, using Protein A sepharose.)

The eluted anti-VASE antibody was used to determine which cell types in the adult rat central nervous system expressed NCAM-VASE. A pan-NCAM antibody recognising all isoforms was used as a comparison to the VASE staining. Staining that is VASE-specific cannot occur in locations that do not stain for NCAM, since VASE-specific staining is part of total NCAM staining.

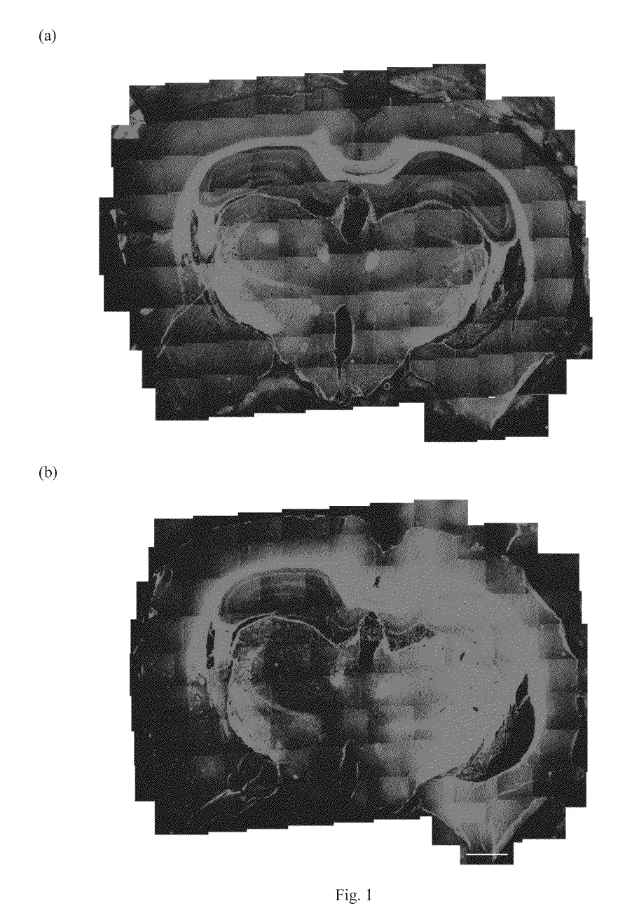

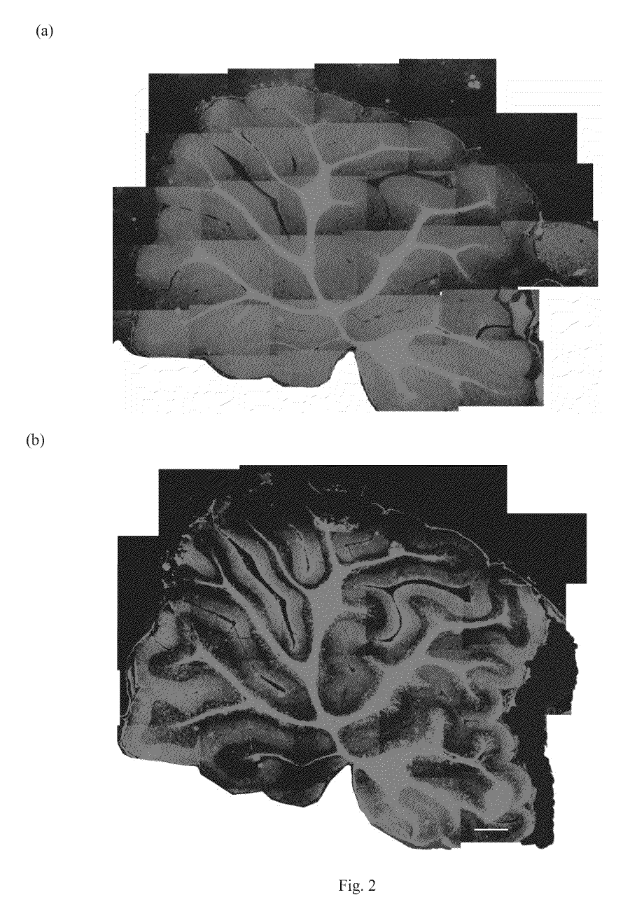

FIG. 1 shows coronal sections of adult rat brain cortex stained for a) total NCAM and b) NCAM-VASE (the bar on FIG. 1 b) equals 600 μm). FIG. 2 shows parasaggital sections of adult rat cerebellum stained for a) total NCAM and b) NCAM-VASE (the bar on FIG. 2 b) equals 600 μm). In both cortex and cerebellum, NCAM-VASE is principally distributed in the white matter tracts, indicating a localisation to myelinated axons.

FIG. 3 shows the localisation of NCAM-VASE in oligodendrocytes (the myelinating cells of the CNS) in the developing rat cerebellum. Expression of myelin marker MOG (left images) is coincident with detection of NCAM-VASE immunoreactivity (middle images), shown in sections from PND7, 10, 14 & 17 rat cerebellum in vivo. NCAM-VASE is also expressed in MOG-expressing oligodendrocytes in culture. This indicates that the white matter localisation of NCAM-VASE is likely to arise from expression in the myelinating oligodendrocytes rather than the axon, although it may be on both. The developmental up-regulation of VASE in oligodendrocytes is also observed when oligodendrocyte precursor cells are isolated by immunopanning with O4 antibody. After two days in vitro (DIV) oligodendrocyte precursors express NCAM but not VASE. However, after 10 DIV they express NCAM-VASE and the myelin marker MOG, also shown in FIG. 3.

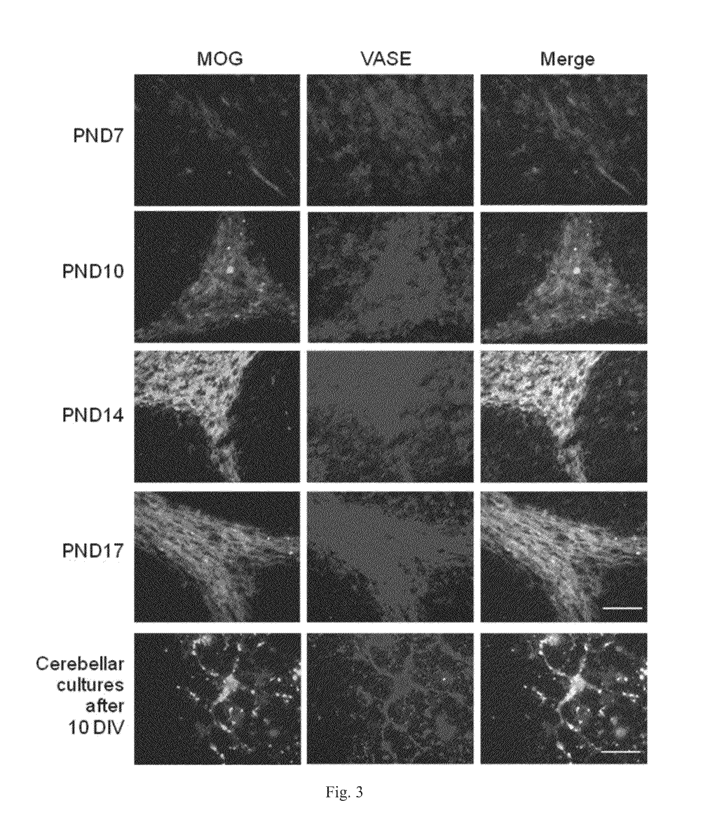

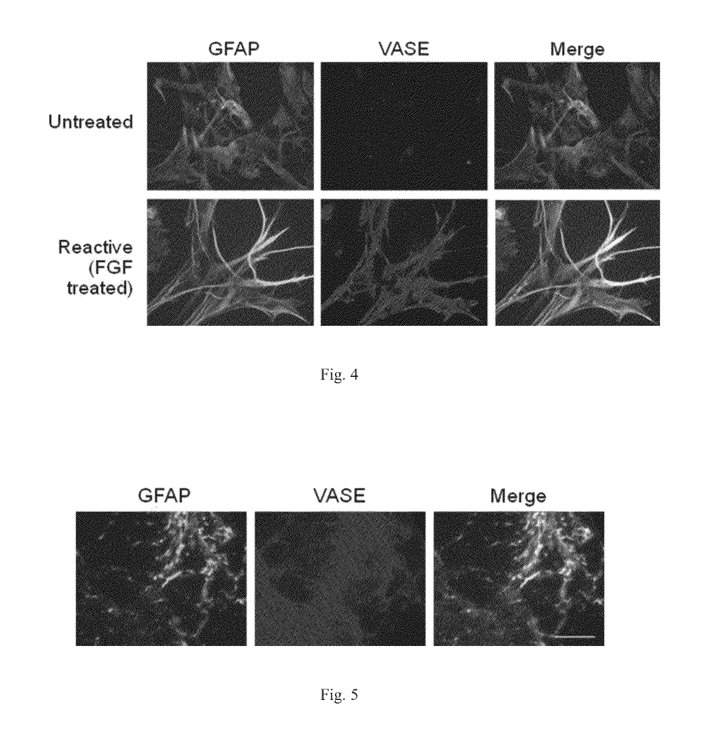

Expression of NCAM-VASE by astrocytes was also examined. Astrocytes (>90% GFAP-positive cells) were isolated from the cortices of PND4 rat and cultured for 2 days in the absence or presence of 1 mM dbcAMP or 50 ng/ml FGF-2 in DMEM/10% FBS. Treatment with dbcAMP or FGF is known to stimulate a reactive phenotype in astrocytes, such as is found in astrocytes in the glial scars that are characteristic of damaged CNS. The astrocytes were then fixed and costained for GFAP and NCAM-VASE (as shown in FIG. 4) or total NCAM (not shown). Astrocytes treated with FGF-2 or dBcAMP showed a large upregulation of total NCAM and NCAM-VASE staining.

NCAM-VASE is also expressed in spinal cord dorsal root entry zone (DREZ) astrocytes, a region known to act as an inhibitory barrier to regeneration of sensory neurons to reinnervate the spinal cord, as shown in FIG. 5.

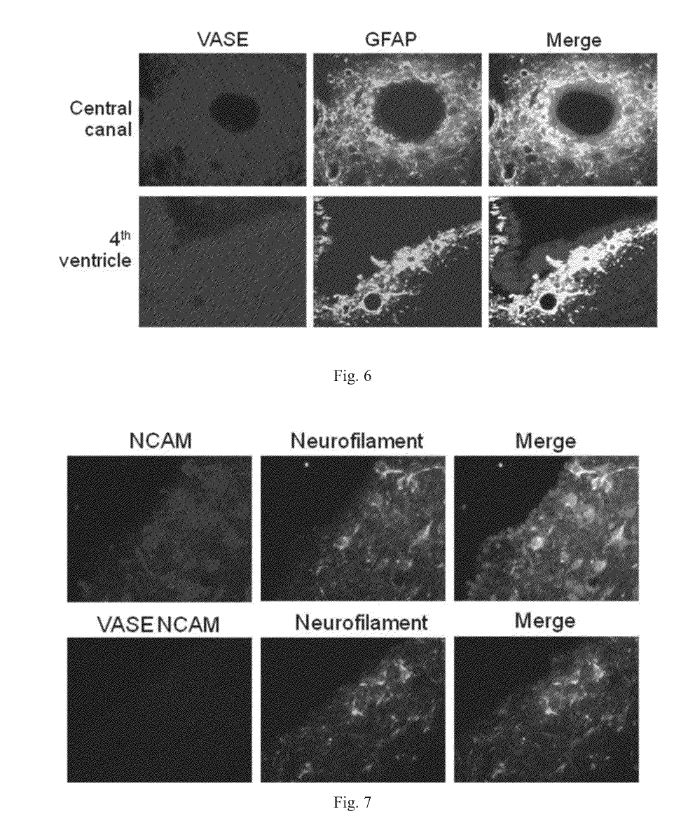

In addition, NCAM-VASE is expressed in the ependymal cells lining the central canal of the spinal cord and the ventricles of the brain, as shown in FIG. 6. NCAM-VASE immunoreactivity (left images) and GFAP immunoreactivity (middle images) are shown, as is DAPI staining which indicates the location of cell nuclei (right, merged, images).

FIG. 7 shows exclusion of NCAM-VASE from ependymal cells lining the lateral ventricle and from NCAM-positive neurons in the sub-ventricular zone. NCAM expression is shown using a pan-NCAM antibody recognising all NCAM isoforms (top left image), and neurofilament expression is also shown (middle images). DAPI staining (right, merged, images) indicates the location of cell nuclei. NCAM (but not NCAM-VASE) is expressed in the neurons in the sub-ventricular zone, but neither VASE nor any other NCAM is expressed in the lateral ventricle ependymal cells. NCAM-VASE is also excluded from the hippocampal dentate gyrus subgranular layer, and from the olfactory bulb (not shown).

### Conclusion:

NCAM-VASE is found in CNS myelin (oligodendrocytes) and reactive astrocytes, tissues known to inhibit CNS regeneration. It could be looked upon as a myelin inhibitory protein, the neutralisation of which may provide a suitable environment for axon regeneration.

### Example 2

### Specific Downregulation of Expression of NCAM-VASE

Use was made of fibroblast clones expressing human non-VASE NCAM (NCAM) or human VASE-containing NCAM (VASE), into which RNAi sequences could be delivered by vector to test the feasibility of targeting VASE specifically. Their effectiveness at down-regulating NCAM/VASE expression was monitored by immunocytochemistry and Western blot.

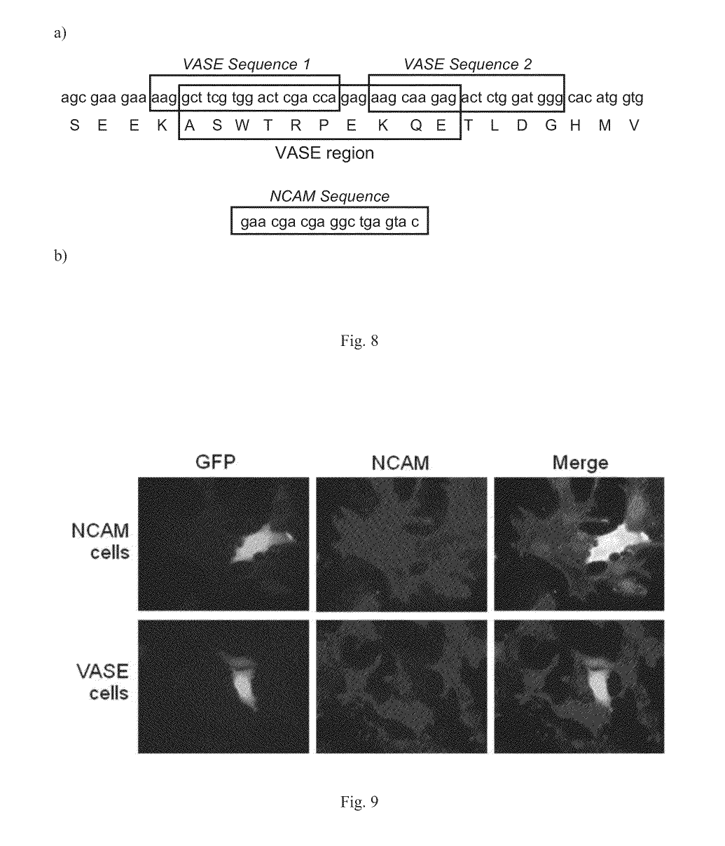

NCAM VASE and non-VASE transcripts are identical apart from the extra 30 bp VASE sequence. This restricts the RNAi target site to these 30 bp. The human VASE-specific sequence was scanned for suitable RNAi sequences, and two possible regions were identified (VASE 1 & VASE 2). In addition, the regions in common between NCAM and VASE were scanned for a suitable control RNAi sequence that would downregulate both NCAM and VASE (pan-NCAM). Target sequences were identified, as shown in FIG. 8:

| | |
|---|---|
| VASE 1: | AAGGCTTCGTGGACTCGACCA (human) |
| VASE 2: | AAGCAAGAGACTCTGGATGGG (human) |
| Pan-NCAM: | GAACGACGAGGCTGAGTAC (human) |

These were each cloned into the pSuperRetro GFP neo OligoEngine vector, transfected into 3T3 VASE- or NCAM-expressing fibroblast clones, and tested for their ability to downregulate expression of human VASE or NCAM protein by immunocytochemistry.

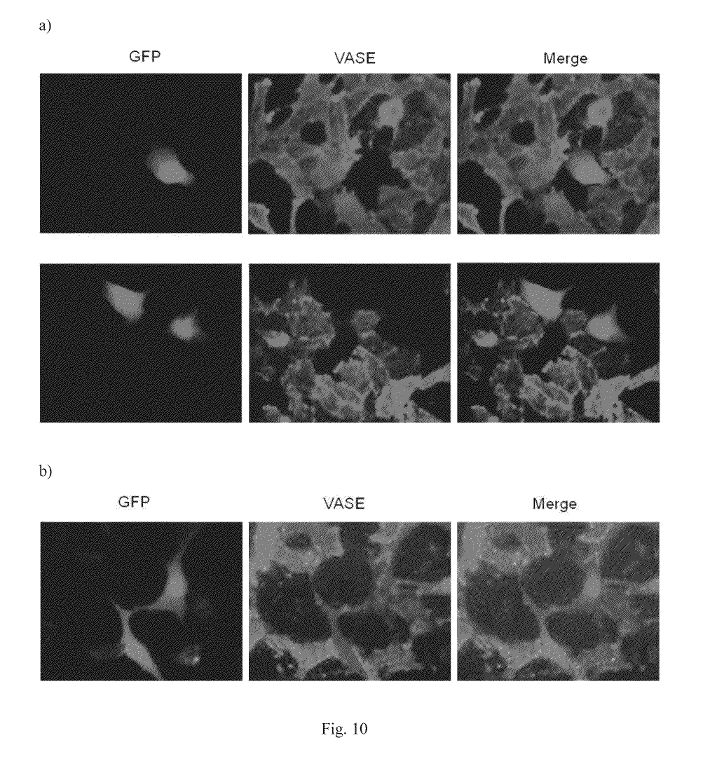

All three sequences were effective at down-regulating levels of their target protein, as shown in FIGS. 9 and 10. The pan-NCAM sequence down-regulated both NCAM-VASE in NCAM-VASE expressing fibroblasts and NCAM in non-VASE NCAM expressing fibroblasts (see FIG. 9). Cells expressing RNAi are identified by co-expression of GFP encoded by the same vector (GFP is shown in the left images). NCAM or NCAM-VASE expression is shown using an antibody that detects an epitope present in each transgene product (middle images).

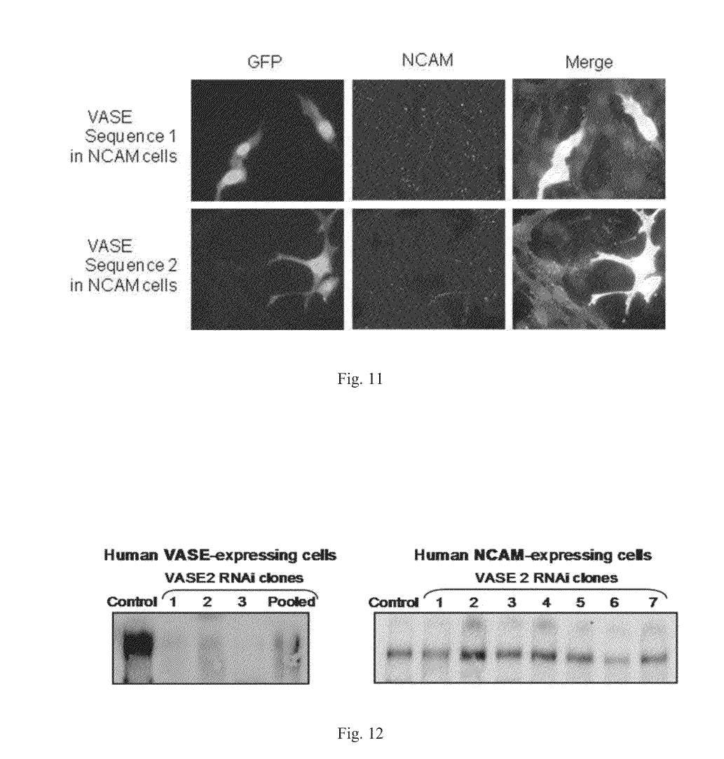

As shown in FIGS. 10 a) and 10 b), NCAM-VASE immunocytochemistry indicated that VASE 1 and VASE 2 sequences both down-regulated expression of VASE specifically. No off-target effect on NCAM expression was observed (see FIG. 11).

The effectiveness of VASE 2 RNAi was further tested by Western blot, as shown in FIG. 12. Cells stably expressing VASE or NCAM transgenes were co-transfected with VASE2 RNAi vector and a puromycin expressing vector. Clones stably expressing either the VASE or NCAM transgene and the RNAi were selected using puromycin, and the levels of NCAM or VASE transgene in each clone assessed by Western blot. FIG. 12 shows a) the lack of VASE expression in three VASE 2 RNAi-expressing clones relative to empty-vector control (in VASE transgene cell background), and b) that in a non-VASE NCAM background, VASE 2 RNAi-expressing clones maintain their NCAM protein expression, i.e. VASE 2 RNAi has no effect on the expression of non-VASE NCAM.

Conclusion: It is possible to selectively down-regulate expression of human VASE without affecting NCAM expression, by targeting the 30 bp human VASE sequence using RNAi.

### Example 3

### Neuronal Contact with VASE-Expressing Cells Prevents Axon Regeneration Stimulated by Soluble NCAM

It has previously been shown that soluble NCAM-Fc stimulates axon outgrowth over control monolayers as effectively as if it were expressed in the monolayer (Meiri et al, J. Neurosci, 1998, 18, 10429-10437). To test whether culture on VASE monolayers suppresses axon outgrowth in the presence of NCAM-Fc, PND4 rat cerebellar granule neurons were cultured on monolayers of 3T3 cells (control) or 3T3 cells stably expressing NCAM or NCAM-VASE overnight in the presence of increasing concentrations of soluble NCAM-Fc (NCAM ectodomain fused to Fc at the C-terminus) before being fixed and stained with a GAP-43 antibody for measurement of mean neurite length. On control monolayers, but not NCAM-VASE-expressing monolayers, soluble NCAM dose-dependently increased mean neurite length.

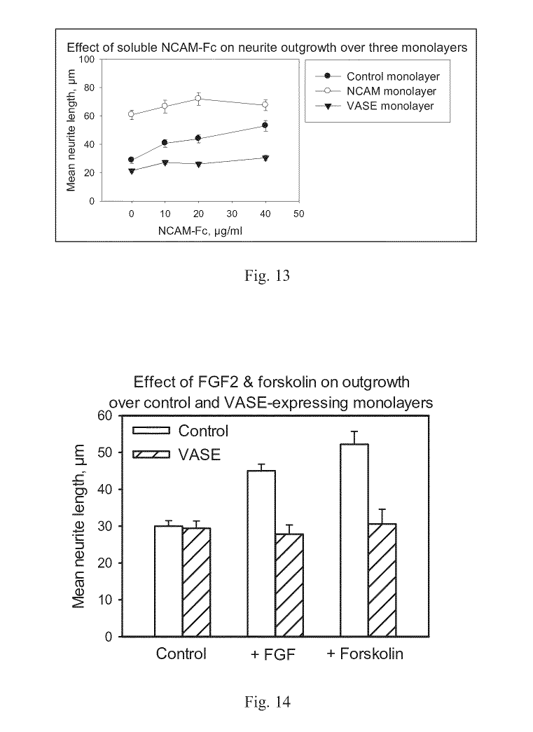

Axon length on control, NCAM- and NCAM-VASE-expressing monolayers in the absence and presence of increasing concentrations of soluble NCAM-Fc was measured. FIG. 13 shows that, consistent with the published data, NCAM-Fc increased axon length over control monolayers. Over NCAM monolayers, the presence of additional (soluble) NCAM-Fc had no augmenting effect on outgrowth (indicating NCAM concentration in monolayer is maximal for outgrowth). Importantly, however, over NCAM-VASE monolayers, NCAM-Fc was unable to stimulate axon outgrowth.

Conclusion: Axon outgrowth stimulated by soluble NCAM is suppressed when neurons are on NCAM-VASE monolayers. The experiment demonstrates that contact with NCAM-VASE cells prevents axon outgrowth even in the presence of non-VASE-NCAM. This indicates the validity of NCAM-VASE as a target for neutralisation, as a strategy for increasing axon regeneration.

### Example 4

### Suppression by NCAM-VASE of Axon Outgrowth Stimulated by Other Outgrowth-Promoting Factors

Axon length on control and NCAM-VASE monolayers in the absence and presence of FGF (0.1 ng/m) or forskolin was measured, both known to stimulate axon outgrowth from cerebellar granule neurons. FIG. 14 shows that cerebellar granule neurons cultured on NCAM-VASE-expressing cells do not extend longer axons when FGF2 or forskolin are added to the culture medium, although both these agents stimulate axon outgrowth from neurons on control monolayers.

Conclusion: Contact with NCAM-VASE-expressing cells inhibits axon outgrowth from neurons, even in the presence of axon outgrowth-stimulating factors that increase axon length when NCAM-VASE is not present. This validates VASE as an important target for neutralisation in strategies aimed at stimulating axon regeneration.

### Example 5

### Cell Contact is Required for VASE Suppression of Axon Outgrowth

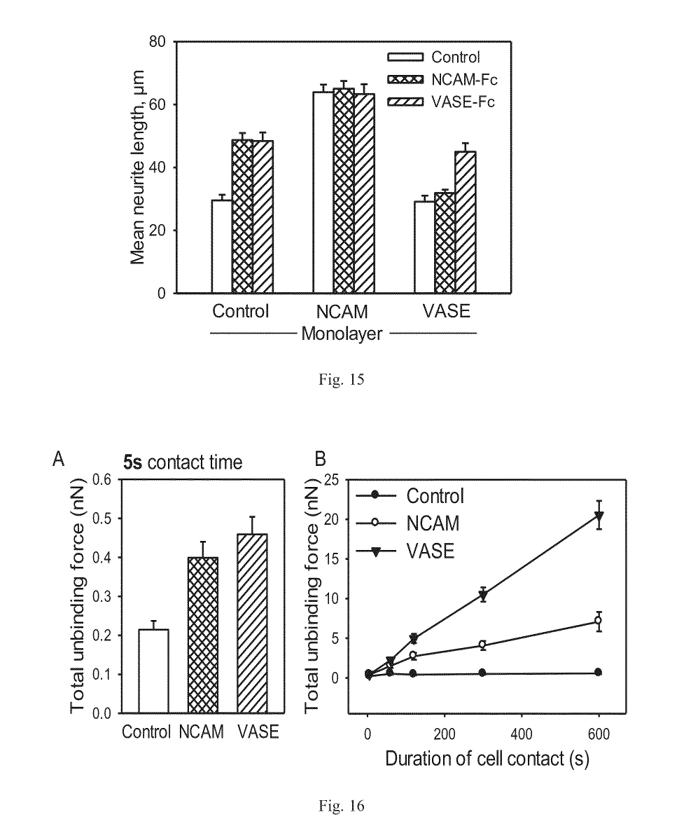

The effect of NCAM-Fc and NCAM-VASE-Fc (both 40 μg/ml) on axon outgrowth over control and NCAM-expressing monolayers was compared. Cerebellar granule neurons were cultured on control, NCAM-expressing (NCAM) or NCAM-VASE-expressing (VASE) monolayers in the absence of presence of soluble NCAM-Fc or NCAM-VASE-Fc (VASE-Fc). Interestingly, NCAM-VASE-Fc was as good as NCAM-Fc at stimulating axon outgrowth over control monolayers, as shown in FIG. 15 (left). Moreover, NCAM-VASE-Fc did not inhibit axon outgrowth stimulated by culture on an NCAM monolayer (FIG. 15, middle). These results show that NCAM-VASE suppression of axon outgrowth requires contact with NCAM-VASE-expressing cells, since soluble NCAM-VASE-Fc does not inhibit axon outgrowth stimulated by NCAM. This is in contrast to VASE peptide, which abolished axon outgrowth stimulated by NCAM (Saffell et al, 1994).

Conclusion: The NCAM-VASE protein is not inherently inhibitory to axon stimulation; its inhibitory activity finds expression only on neuronal contact with NCAM-VASE-expressing cells. Soluble NCAM ectodomains containing VASE do not inhibit NCAM stimulated axon outgrowth, and in fact can stimulate same.

### Example 6

### Soluble NCAM-VASE-Fc Relieves the Inhibition of Axon Outgrowth Resulting from Contact with NCAM-VASE-Expressing Cells

FIG. 15 shows that, in the presence of soluble NCAM-VASE-Fc, outgrowth over NCAM-VASE monolayers increased. This suggests that the contact-mediated suppression of outgrowth mediated by substratum-expressed NCAM-VASE can be displaced by a soluble NCAM-VASE-Fc, which presumably competes with cell-associated NCAM-VASE for adhesion to neurons. In this experiment, NCAM-VASE-Fc is presumably playing two roles: first, competing with monolayer NCAM-VASE to neutralise its inhibition of axon regeneration, and second, its first three Ig domains may be binding to neuronal NCAM to stimulate axon outgrowth. (The first three Ig domains of NCAM have been shown to be sufficient to stimulate axon outgrowth equivalent to that stimulated by the whole NCAM ectodomain, Meiri et al, J. Neurosci, 1998, 18, 10429-10437)

Conclusion: Soluble NCAM VASE-Fc relieves contact-mediated NCAM-VASE suppression of axon outgrowth and has therapeutic potential for use in CNS repair following damage/disease.

### Example 7

### NCAM-VASE Exerts Stronger Homophilic Cell Adhesion Force than NCAM

JPK CellHesion 200 single cell force spectroscopy equipment was used to compare the force required to detach homophilic binding of cells expressing no NCAM, NCAM or NCAM-VASE (equal cell surface expression level). In brief, rounded cells in suspension were attached to a fibronectin-coated cantilever and brought into apposition with cells cultured overnight as an ‘island’ in the centre of the dish. After variable contact times (5-600 seconds), the cantilever with attached cell was raised, and the force required to detach the cells measured. The long pulling range (100 μm piezo movement) makes complete detachment of the cells possible over extended cell-cell contact times. FIG. 16 shows the force required to detach homophilic binding of cells expressing no NCAM, NCAM or NCAM-VASE following a) 5 seconds contact time between cells, and b) following variable contact times between cells. FIG. 16 a) shows that after 5 seconds homophilic contact, twice as much force was required to pull apart NCAM and NCAM-VASE cells than control cells, but there was no difference between NCAM and NCAM-VASE. However, after longer contact times (5, 60, 120, 300 and 600 seconds), the force required to detach two NCAM-VASE expressing cells from one another was considerably greater that required to detatch NCAM-expressing cells (as shown in FIG. 16 b)).

Conclusion: NCAM-VASE mediates much stronger cell-cell adhesion than does NCAM. Failure of NCAM-VASE to stimulate axon outgrowth is not a consequence of poor homophilic binding.

1. (canceled)

2. (canceled)

3. The method of claim 12 wherein the inhibitor is an antisense RNA molecule or an interfering RNA that is capable of causing a reduction of NCAM-VASE mRNA transcription.

4. The method of claim 12 wherein the inhibitor is a soluble polypeptide comprising the Ig4 domain of NCAM-VASE.

5. The method of claim 4 wherein the inhibitor is a soluble polypeptide comprising a further sequence from NCAM-VASE in addition to the Ig4 domain.

6. The method of claim 12 wherein the inhibitor is an antibody or antibody fragment or derivative able to bind selectively to NCAM-VASE.

7. The method of claim 12, wherein the inhibitor is administered in combination with a second agent which promotes neuroplasticity and/or neuroregeneration.

8. The method of claim 7, wherein the second agent is selected from the list consisting of NGF, BDNF, FGF, CNTF, GDNF, NT3, NT4/5 dbcAMP and forskolin.

9. The method of claim 12, wherein the method is for treating brain or spinal cord injury.

10. The method of claim 12, wherein the method is for treating schizophrenia, motor neurone disease, for treating a neurodegenerative disorder including Alzheimer's disease, multiple sclerosis or Parkinson's disease; for treating ischaemia caused by stroke; or for improving learning and/or memory.

11. (canceled)

12. A method of stimulating in vitro or in vivo neuroplasticity and/or neuroregeneration comprising providing an inhibitor of NCAM-VASE to neuronal cells and/or tissues.

13. A method of enhancing neuroplasticity and/or neuroregeneration in a human subject in need thereof comprising providing a therapeutically effective amount of an inhibitor of NCAM-VASE to said subject.

14. The method of claim 13 wherein said method is for treating a brain or spinal injury.

15. The method of claim 13 wherein said method is for treating schizophrenia, motor neurone disease, for treating a neurodegenerative disease, for example Alzheimer's disease, multiple sclerosis, or Parkinson's disease; for treating ischaemia caused by stroke; or for improving learning and/or memory.

16. (canceled)

17. A compound which inhibits NCAM-VASE but which does not inhibit other isoforms of NCAM, said inhibitor selected from the group consisting of, an anti-sense nucleic acid or an interfering nucleic acid capable to selectively reducing the expression of an mRNA having the nucleotide sequence: gcttcgtggactcgaccagagaagcaagag (SEQ ID NO: 1); an antibody or derivative thereof able to bind to NCAM-VASE; and a soluble protein comprising the Ig4 domain of NCAM-VASE.

18. (canceled)

19. (canceled)

20. (canceled)

21. A pharmaceutical composition comprising an inhibitor of NCAM-VASE as recited in claim 17.

22. The method of claim 13, wherein the inhibitor is an antisense RNA molecule or an interfering RNA that is capable of causing a reduction of NCAM-VASE mRNA transcription.

23. The method of claim 13, wherein the inhibitor is an is a soluble polypeptide comprising the Ig4 domain of NCAM-VASE.

24. The method of claim 13, wherein the inhibitor is a soluble polypeptide comprising further sequence from NCAM-VASE in addition to the Ig4 domain.

25. The method of claim 13, wherein the inhibitor is an antibody or antibody fragment or derivative able to bind selectively to NCAM-VASE.

26. The method of claim 13, wherein the inhibitor is administered in combination with a second agent which promotes neuroplasticity and/or neuroregeneration.

27. The method of claim 26, wherein the second agent is selected from the list consisting of NGF, BDNF, FGF, CNTF, GDNF, NT3, NT4/5 dbcAMP and forskolin.


SEQUENCE LISTING (10 sequences)

SEQ ID NO: 1 - DNA, 30 bp, Homo sapiens
gcttcgtggactcgaccagagaagcaagag

SEQ ID NO: 2 - protein, 10 aa, Homo sapiens
ASWTRPEKQE

SEQ ID NO: 3 - DNA, 30 bp, Rattus rattus
gcatcgtggactcgaccagagaagcaagag

SEQ ID NO: 4 - protein, 10 aa, Rattus rattus
ASWTRPEKQE

SEQ ID NO: 5 - protein, 19 aa, Artificial sequence
  synthetic peptide immunogen
CSEEKASWTRPEKQETLDG

SEQ ID NO: 6 - DNA, 21 bp, Homo sapiens
aaggcttcgtggactcgacca

SEQ ID NO: 7 - DNA, 21 bp, Homo sapiens
aagcaagagactctggatggg

SEQ ID NO: 8 - DNA, 19 bp, Homo sapiens
gaacgacgaggctgagtac

SEQ ID NO: 9 - DNA, 63 bp, Homo sapiens
agcgaagaaaaggcttcgtggactcgaccagagaagcaagagactctggatgggcacatggtg

SEQ ID NO: 10 - protein, 21 aa, Homo sapiens
SEEKASWTRPEKQETLDGHMV